SEQUENCE LISTING (8 sequences)

SEQ ID NO: 1 - DNA, 27 bp, Homo sapiens
accgactccagctcccgaaacgaagag

SEQ ID NO: 2 - DNA, 23 bp, Homo sapiens
tgaagagatacgccctggttcct

SEQ ID NO: 3 - DNA, 25 bp, Homo sapiens
gtgtcgtggagtcggcaaaggaacc

SEQ ID NO: 4 - DNA, 20 bp, Homo sapiens
cgcgctagcagcacgtaaat

SEQ ID NO: 5 - DNA, 16 bp, Homo sapiens
atacgaccgccaatat

SEQ ID NO: 6 - DNA, 13 bp, Homo sapiens
agcctgggacgtg

SEQ ID NO: 7 - DNA, 50 bp, Homo sapiens
aaccgccagcataggtcacgcttatggagcctgggacgtgacctatgctg

SEQ ID NO: 8 - RNA, 22 nt, Homo sapiens
uagcagcacguaaauauuggcg

(19) United States
(12) Patent Application Publication
(10) Pub. No.: US 2007/0054287 A1
(43) Pub. Date: Mar. 8, 2007

(54) METHOD FOR IDENTIFYING MEDICALLY IMPORTANT CELL POPULATIONS USING MICRO RNA AS TISSUE SPECIFIC BIOMARKERS

(75) Inventor: Will Bloch, White Salmon, WA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/421,456

(22) Filed: May 31, 2006

Related U.S. Application Data
(60) Provisional application No. 60/686,274, filed on May 31, 2005.

(51) Int. Cl. C12Q 1/68; C12P 19/34

(52) U.S. Cl. 435/6; 435/91.2

(57) ABSTRACT

The present teachings provide methods for diagnosing biological conditions, including cancer. In some embodiments, a test sample is collected from a subject such as a clinical patient, wherein the test sample comprises background tissue and may or may not contain cells from a tissue of interest. Observation of a target miRNA normally present in a tissue of interest, but collected in an anatomical location ectopic to the tissue of interest, can be indicative of a biological condition. The present teachings further provide exponential amplification techniques applicable to performing these analyses.

### CROSS REFERENCE TO RELATED APPLICATIONS

This application claims a priority benefit under 35 U.S.C. § 119(e) from U.S. Patent Application No. 60/686,274, filed May 31, 2005, which is incorporated herein by reference in its entirety.

### FIELD

The present teachings generally relate to methods for detecting biological conditions such as cancer by using micro RNAs as tissue-specific biomarkers.

### INTRODUCTION

Despite recent advances, modern medicine continues to lack methods for accurate and sensitive cellular identification (see for example U.S. Pat. No. 6,441,269). For example cancer diagnosis and prognosis continues to lack assays of sufficient speed, accuracy, sensitivity, and dynamic range. While the central dogma of molecular biology maintains that DNA codes for messenger RNA, which in turn encodes protein, increasing evidence indicates an important role for small RNA molecules termed micro RNAs (micro RNAs) in regulating gene expression. Published functions of micro RNAs are numerous, and include control of cell proliferation, cell death, and fat metabolism in flies (Brennecke et al., 2003, Cell, 113 (1), 25-36; Xu et al, 2003, Current Biology, 13 (9), 790-795), neuronal patterning in nematodes (Johnston and Hobert, 2003, Nature, 426 (6968), 845-849), modulation of hematopoietic lineage differentiation in mammals (Chen et al., 2004, Science, 303 (5654), 83-87), and control of leaf and flower development in plants (Aukerman and Sakai, 2003, Plant Cell, 15 (11), 2730-2741; Chen, 2003, Science, 303 (5666):2022-2025; Emery et al., 2003, Current Biology, 13 (20), 1768-1774; Palatnik et al., 2003, Nature, 425 (6955), 257-263).

### SUMMARY

In some embodiments, the present teachings provide a method for diagnosing a biological condition comprising; amplifying a target micro RNA from a test sample to provide a derived micro RNA quantity, wherein the target micro RNA is from a tissue of interest; comparing the derived micro RNA quantity to an expectation micro RNA quantity from a background tissue; and, diagnosing the biological condition.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts certain aspects of various compositions according to some embodiments of the present teachings.

FIG. 2 depicts certain aspects of various compositions according to some embodiments of the present teachings.

FIG. 3 depicts certain sequences of various compositions according to some embodiments of the present teachings.

FIG. 4 depicts one single-plex assay design according to some embodiments of the present teachings.

FIG. 5 depicts an overview of a multiplex assay design according to some embodiments of the present teachings.

FIG. 6 depicts a multiplex assay design according to some embodiments of the present teachings.

FIG. 7 depicts certain sequences of various compositions according to some embodiments of the present teachings.

FIG. 8 depicts certain sequences of various compositions according to some embodiments of the present teachings.

FIG. 9 depicts an overview of assessing a tissue-specific micro RNA in the context of a clinical setting.

### DESCRIPTION OF EXEMPLARY EMBODIMENTS

Aspects of the present teachings may be further understood in light of the following examples, which should not be construed as limiting the scope of the present teachings in any way. The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. For example, “a primer” means that more than one primer can, but need not, be present; for example but without limitation, one or more copies of a particular primer species, as well as one or more versions of a particular primer type, for example but not limited to, a multiplicity of different forward primers. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

Some Definitions

As used herein, the term “target micro RNA” is used to refer to a micro RNA that is expressed in a tissue of interest, and not expressed (or expressed to a significantly lesser extent) in a background tissue. In some embodiments, a target micro RNA is expressed in a background tissue, and not expressed (or expressed to a significantly lesser extent) in a tissue of interest.

As used herein, the term “test sample” refers to a collection of molecules that contains or is derived from a background tissue, and potentially a tissue of interest. For example, a test sample of blood can be collected from a patient and a target micro RNA is quantified. The present teachings contemplate embodiments in which a test aliquot (part of the test sample) is obtained from the blood, as well as embodiments in which the test aliquot is the entirety of the test sample (e.g. all of the blood in the test sample). Test samples can be collected by a number of procedures, including but not limited to needle aspirates and biopsies, peritoneal fluid, surgical explants and scrapings, histological sections, cytospin preparations, cell isolated by laser-capture microdissection, cells isolated by magnetically activated cell sorting (MACS), cell isolated by fluorescently activated cells sorting (FACS), cells isolated by immunoprecipitation, cell isolated by immunopanning, intravenous blood draws, finger sticks, and various swabs, including buccal.

As used herein, the term “background tissue” refers to at least one tissue that is not a tissue of interest, and which differs in the expression of at least one target micro RNA as compared to a tissue of interest. As used herein, the term “tissue of interest” refers to at least one tissue that is not a background tissue, and which differs in the expression of at least one target micro RNA as compared to a background tissue. Typically, in the context of cancer diagnosis, a test sample will be collected that largely comprises background tissue present at its normal anatomical location, and the test sample may further comprise a tissue of interest that can be present at a site ectopic to that tissue of interest's normal anatomical location. However, it will be appreciated that the terms “background tissue” and “tissue of interest” are relative terms, and serve the function of orienting the reader to a particular context.

As used herein, the term “expectation micro RNA quantity” refers to a quantity that can be compared to for the purposes of determining the actual quantity of the target micro RNA in the test sample. For example, a known amount of a micro RNA that is normally present in a particular kind of test sample, for example a healthy test sample, can be used as an expectation micro RNA quantity. In such a scenario, a given amount of test sample (e.g.—grams of tissue, number of cells) known to comprise the target micro RNA in a known quantity, can be used as an expectation micro RNA quantity to compare to the quantity of target micro RNA present in the test sample under inquiry. Such an expectation micro RNA quantity can be determined in a parallel reaction, for example a parallel reaction comprising a sample collected exclusively from a background tissue. In some embodiments, the expectation micro RNA quantity can be known from the scientific literature. For example, the quantity of target micro RNA can be known for a tissue of interest, or for a background tissue. In some embodiments, the target micro RNA present in the background tissue can itself be an endogenous internal control (see below), and compared to the target micro RNA quantity present in the tissue of interest.

As used herein, the term “endogenous control small RNA” refers to a small RNA that is present in the test sample and used to normalize the quantity of target micro RNA in the test sample. Thus, the endogenous control small RNA can be used to normalize the quantity of target micro RNA in the test sample itself, thus accounting for variability in reaction efficiency and/or sample input. That is, before determination of a biological condition by comparing a derived micro RNA quantity to an expectation micro RNA quantity, logically one must first determine what the derived micro RNA quantity is. Thus, the endogenous control small RNA can be employed to determine the derived micro quantity. In some embodiments, the endogenous control RNA is queried in a parallel reaction mixture to the reaction mixture querying the target micro RNA, wherein both reaction mixtures contain an aliquot of the same test sample. In some embodiments, the endogenous control RNA is queried in the same reaction mixture where the target micro RNA is being queried, and can be considered an internal endogenous control RNA. In some embodiments, an internal control can be employed that is not an endogenous small RNA, for example a synthetic molecule of known concentration can be added to the reaction containing the target micro RNA, and the quantity of the target micro RNA determined by comparison to the signal derived from the synthetic molecule. In some embodiments, a synthetic molecule of known concentration can be analyzed in a parallel reaction. In some embodiments, the endogenous control small nucleic acid, and/or the internal controls, are micro RNAs.

As used herein, the phrase “diagnosing a biological condition” can refer to any of a variety of conclusions drawn from the quantitation of at least one target micro RNA in a test sample. For example, in a scenario where blood is drawn from a patient and a prostate-specific micro RNA is quantified, the diagnosing a biological condition can be indicative of a metastatic prostate cancer. Analogously, in a scenario where blood is drawn and a breast-specific micro RNA is quantified, the diagnosing a biological condition can be indicative of the presence of a metastatic breast cancer, or the absence of a metastatic breast cancer. In a scenario where the test sample comprises stem cells, quantitation of a target micro RNA can be indicative of the level of purity of the stem cells, and/or the level of differentiation of stem cells treated with a reagent intended to induce differentiation into a tissue of interest. In some embodiments, the diagnosing a biological condition comprises monitoring for minimal residual disease after initial therapy, especially in various cancers.

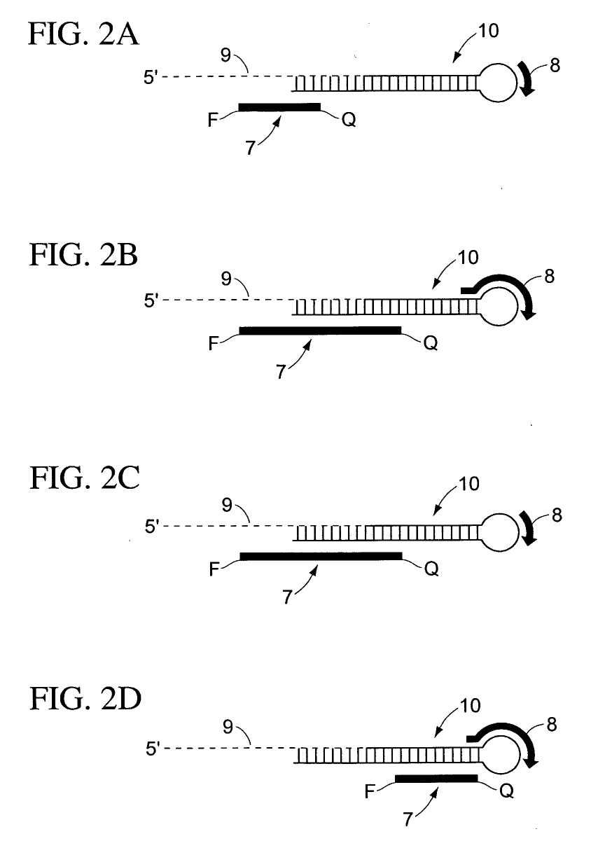

As used herein, the term “stem-loop primer” refers to a molecule comprising a 3′ target specific portion, a stem, and a loop. Illustrative stem-loop primers are depicted in FIG. 2, elsewhere in the present teachings, and in U.S. patent application Ser. No. 10/947,460 to Chen et al., and co-filed U.S. Non-Provisional Patent Application Methods for Characterizing Cells Using Amplified Micro RNAs claiming priority to U.S. Provisional Application 60/686,521, and 60/708,946. The term “3′ target-specific portion” refers to the single stranded portion of a stem-loop primer that is complementary to a target polynucleotide such as target micro RNA or endogenous control small RNA. The 3′ target-specific portion is located downstream from the stem of the stem-loop primer. Generally, the 3′ target-specific portion is between 6 and 8 nucleotides long. In some embodiments, the 3′ target-specific portion is 7 nucleotides long. It will be appreciated that routine experimentation can produce other lengths, and that 3′ target-specific portions that are longer than 8 nucleotides or shorter than 6 nucleotides are also contemplated by the present teachings. Generally, the 3′-most nucleotides of the 3′ target-specific portion should have minimal complementarity overlap, or no overlap at all, with the 3′ nucleotides of the forward primer; it will be appreciated that overlap in these regions can produce undesired primer dimer amplification products in subsequent amplification reactions. In some embodiments, the overlap between the 3′-most nucleotides of the 3′ target-specific portion and the 3′ nucleotides of the forward primer is 0, 1, 2, or 3 nucleotides. In some embodiments, greater than 3 nucleotides can be complementary between the 3′-most nucleotides of the 3′ target-specific portion and the 3′ nucleotides of the forward primer, but generally such scenarios will be accompanied by additional non-complementary nucleotides interspersed therein. In some embodiments, modified bases such as LNA can be used in the 3′ target specific portion to increase the Tm of the stem-loop primer (see for example Petersen et al., Trends in Biochemistry (2003), 21:2:74-81). In some embodiments, universal bases can be used, for example to allow for smaller libraries of stem-loop primers. In some embodiments, modifications including but not limited to LNAs and universal bases can improve reverse transcription specificity and potentially enhance detection specificity. The term “stem” refers to the double stranded region of the stem-loop primer that is between the 3′ target-specific portion and the loop. Generally, the stem is between 6 and 20 nucleotides long (that is, 6-20 complementary pairs of nucleotides, for a total of 12-40 distinct nucleotides). In some embodiments, the stem is 8-14 nucleotides long. As a general matter, in those embodiments in which a portion of the detector probe is encoded in the stem, the stem can be longer. In those embodiments in which a portion of the detector probe is not encoded in the stem, the stem can be shorter. Those in the art will appreciate that stems shorter than 6 nucleotides and longer than 20 nucleotides can be identified in the course of routine methodology and without undue experimentation, and that such shorter and longer stems are contemplated by the present teachings. In some embodiments, the stem can comprise an identifying portion. The term “loop” refers to a region of the stem-loop primer that is located between the two complementary strands of the stem, as depicted for example in FIG. 2. Typically, the loop comprises single stranded nucleotides, though other moieties including modified DNA or RNA, Carbon spacers such as C18, and/or PEG (polyethylene glycol) are also possible. Generally, the loop is between 4 and 20 nucleotides long. In some embodiments, the loop is between 14 and 18 nucleotides long. In some embodiments, the loop is 16 nucleotides long. As a general matter, in those embodiments in which a reverse primer is encoded in the loop, the loop can generally be longer. In those embodiments in which the reverse primer corresponds to both the target polynucleotide as well as the loop, the loop can generally be shorter. Those in the art will appreciate that loops shorter that 4 nucleotides and longer than 20 nucleotides can be identified in the course of routine methodology and without undue experimentation, and that such shorter and longer loops are contemplated by the present teachings. In some embodiments, the loop can comprise an identifying portion, also known as a “zipcode.”

As used herein, the term “forward primer” refers to a primer in an amplification reaction such as PCR, as readily known by one of skill in the art of molecular biology (see for example Sambrook and Russell, Molecular Cloning, 3^{rd} Edition).

In some embodiments of the present teachings, for example when used in conjunction with stem-loop primers, the forward primer comprises an extension reaction product portion and a tail portion. The extension reaction product portion of the forward primer hybridizes to the extension reaction product. Generally, when used in conjunction with stem-loop primers, the extension reaction product portion of the forward primer is between 9 and 19 nucleotides in length. The tail portion is located upstream from the extension reaction product portion, and is not complementary with the extension reaction product; after a round of amplification however, the tail portion can hybridize to complementary sequence of amplification products. Generally, when used in conjunction with stem-loop primers, the tail portion of the forward primer is between 5-8 nucleotides long. Those in the art will appreciate that forward primer tail portion lengths shorter than 5 nucleotides and longer than 8 nucleotides can be identified in the course of routine methodology and without undue experimentation, and that such shorter and longer forward primer tail portion lengths are contemplated by the present teachings. Further, those in the art will appreciate that lengths of the extension reaction product portion of the forward primer shorter than 9 nucleotides in length and longer than 19 nucleotides in length can be identified in the course of routine methodology and without undue experimentation, and that such shorter and longer extension reaction product portion of forward primers are contemplated by the present teachings.

As used herein, the term “reverse primer” refers to a primer in an amplification reaction such as PCR, as readily known by one of skill in the art of molecular biology (see for example Sambrook and Russell, Molecular Cloning, 3^{rd} Edition).

In some embodiments of the present teachings, for example when used in conjunction with stem-loop primers, the reverse primer corresponds with a region of the loop of a stem-loop primer. Following the extension reaction with the stem-loop reverse primer, the forward primer can hybridize to the extension product and can be extended to form a second strand product. The reverse primer hybridizes with this second strand product, and can be extended to continue the amplification reaction. In some embodiments, the reverse primer corresponds with a region of the loop of a stem-loop primer, a region of the stem of a stem-loop primer, and/or a region of the target polynucleotide. Generally, the reverse primer when used in conjunction with stem-loop primers is between 13-16 nucleotides long. In some embodiments, the reverse primer can further comprise a non-complementary tail region, though such a tail is not required. In some embodiments, the reverse primer is a “universal reverse primer,” which indicates that the sequence of the reverse primer can be used in a plurality of different reactions querying different target polynucleotides, but that the reverse primer nonetheless is the same sequence.

As used herein, the term “ligation probe” refers to a polynucleotide used in a ligation reaction to query a target polynucleotide. Typically, a ligation reaction will comprise a first ligation probe and a second ligation probe, which upon hybridization to a target polynucleotide, can be ligated together. Illustrative ligation probes and their use can be found, for example, in Published U.S. application Ser. No. 03/308891 to Wenz et al., U.S. Pat. No. 6,797,470 to Barany et al., and U.S. Pat. No. 6,511,810 to Bi et al., and U.S. Non-Provisional patent application Ser. No. 10/881,362 to Karger et al.,

It will be appreciated that the stem-loop primers, ligation probes, and the primers of the present teachings, can be comprised of ribonucleotides, deoxynucleotides, modified ribonucleotides, modified deoxyribonucleotides, modified phosphate-sugar-backbone oligonucleotides, nucleotide analogs, or combinations thereof. For some illustrative teachings of various nucleotide analogs etc, see Fasman, 1989, Practical Handbook of Biochemistry and Molecular Biology, pp. 385-394, CRC Press, Boca Raton, Fla., Loakes, N.A.R. 2001, vol 29:2437-2447, and Pellestor et al., Int J Mol Med. 2004 April; 13(4):521-5.), references cited therein, and any recent articles citing these reviews. It will be appreciated that the selection of the stem-loop primers, primers, and ligation probes to query a given target micro RNA, and the selection of which collection of target micro RNA s to query in a given reaction will involve procedures generally known in the art, and can involve the use of algorithms to select for those sequences with minimal secondary and tertiary structure, those targets with minimal sequence redundancy with other regions of the genome, those target regions with desirable thermodynamic characteristics, and other parameters desirable for the context at hand. Further, it will be appreciated that formation of nonspecific amplification products during PCR is a common problem in molecular biology. Such nonspecific products can be formed due to nonspecific primer/template and/or primer/primer annealing events. These events can provide substrate for the DNA polymerase. Any products formed in this manner can be templates for subsequent amplification, resulting in nonspecific products and/or primer-dimer formation. A number of steps may be taken to reduce the formation of nonspecific products, such as, optimal concentration of salts and others components, optimal temperature regimen, hot start, additives etc. The present teachings further contemplate embodiments including the presence of modified nucleotides in primer sequence to reduce primer dimer formation, as taught for example in U.S. Non-Provisional patent application Ser. No. 11/106,044 to Ma and Mullah.

As used herein, the term “detector probe” refers to a molecule used in an amplification reaction, typically for quantitative or real-time PCR analysis, as well as end-point analysis. Such detector probes can be used to monitor the amplification of the target micro RNA and/or control nucleic acids such as endogenous control small nucleic acids and/or synthetic internal controls. In some embodiments, detector probes present in an amplification reaction are suitable for monitoring the amount of amplicon(s) produced as a function of time. Such detector probes include, but are not limited to, the 5′-exonuclease assay (TaqMan® probes described herein (see also U.S. Pat. No. 5,538,848) various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:408-84093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can also comprise quenchers, including without limitation black hole quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher is on the other probe, wherein hybridization of the two probes together on a target quenches the signal, or wherein hybridization on the target alters the signal signature via a change in fluorescence. Illustrative detector probes comprising two probes wherein one molecule is an L-DNA and the other molecule is a PNA can be found in U.S. Provisional Application 60/584,799 to Lao et al., Detector probes can also comprise sulfonate derivatives of fluorescenin dyes with SO3 instead of the carboxylate group, phosphoramidite forms of fluorescein, phosphoramidite forms of CY 5 (commercially available for example from Amersham). In some embodiments, intercalating labels are used such as ethidium bromide, SYBR® Green I (Molecular Probes), and PicoGreen® (Molecular Probes), thereby allowing visualization in real-time, or end point, of an amplification product in the absence of a detector probe. In some embodiments, real-time visualization can comprise both an intercalating detector probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, probes can further comprise various modifications such as a minor groove binder (see for example U.S. Pat. No. 6,486,308) to further provide desirable thermodynamic characteristics. In some embodiments, detector probes can correspond to identifying portions or identifying portion complements, also referred to as zip-codes. Descriptions of identifying portions can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); 5,935,793 (referred to as “identifier tags” therein); and PCT Publication No. WO 01/92579 (referred to as “addressable support-specific sequences” therein).

The term “corresponding” as used herein refers to a specific relationship between the elements to which the term refers. Some non-limiting examples of corresponding include: a stem-loop primer can correspond with a target polynucleotide such a target micro RNA, and vice versa. A forward primer can correspond with a target polynucleotide such as a target micro RNA, and vice versa. A stem-loop primer can correspond with a forward primer for a given target polynucleotide such as a target micro RNA, and vice versa. The 3′ target-specific portion of the stem-loop primer can correspond with the 3′ region of a target polynucleotide such as a target micro RNA, and vice versa. A detector probe can correspond with a particular region of a target polynucleotide such as a target micro RNA and vice versa. A detector probe can correspond with a particular identifying portion and vice versa. In some cases, the corresponding elements can be complementary. In some cases, the corresponding elements are not complementary to each other, but one element can be complementary to the complement of another element.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “detection” refers to any of a variety of ways of determining the presence and/or quantity and/or identity of a target polynucleoteide. In some embodiments employing a donor moiety and signal moiety, one may use certain energy-transfer fluorescent dyes. Certain nonlimiting exemplary pairs of donors (donor moieties) and acceptors (signal moieties) are illustrated, e.g., in U.S. Pat. Nos. 5,863,727; 5,800,996; and 5,945,526. Use of some combinations of a donor and an acceptor have been called FRET (Fluorescent Resonance Energy Transfer). In some embodiments, fluorophores that can be used as signaling probes include, but are not limited to, rhodamine, cyanine 3 (Cy 3), cyanine 5 (Cy 5), fluorescein, Vic™, LiZ™, Tamra™, 5-Fam™, 6-Fam™, and Texas Red (Molecular Probes). (Vic™, LiZ™, Tamra™, 5-Fam™, and 6-Fam™ (all available from Applied Biosystems, Foster City, Calif.). In some embodiments, the amount of detector probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator. According to some embodiments, one can employ an internal standard to quantify the amplification product indicated by the fluorescent signal, see, e.g., U.S. Pat. No. 5,736,333, and infra in the present teachings. Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670, and include, but are not limited to the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), and the ABI GeneAmp® 7500 Sequence Detection System (Applied Biosystems). In some embodiments, each of these functions can be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product. In some embodiments, combined thermal cycling and fluorescence detecting devices can be used for precise quantitation of target nucleic acid sequences in samples. In some embodiments, fluorescent signals can be detected and displayed during and/or after one or more thermal cycles, thus permitting monitoring of amplification products as the reactions occur in “real time.” In some embodiments, one can use the amount of amplification product and number of amplification cycles to calculate how much of the target nucleic acid sequence was in the sample prior to amplification. In some embodiments, one could simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target polynucleotide. As used herein, determining the presence of a target can comprise identifying it, as well as optionally quantifying it. In some embodiments, the amplification products can be scored as positive or negative as soon as a given number of cycles is complete. In some embodiments, the results may be transmitted electronically directly to a database and tabulated. Thus, in some embodiments, large numbers of samples can be processed and analyzed with less time and labor when such an instrument is used. In some embodiments, different detector probes may distinguish between different target polynucleotides. A non-limiting example of such a probe is a 5′-nuclease fluorescent probe, such as a TaqMan® probe molecule, wherein a fluorescent molecule is attached to a fluorescence-quenching molecule through an oligonucleotide link element. In some embodiments, the oligonucleotide link element of the 5′-nuclease fluorescent probe binds to a specific sequence of an identifying portion or its complement. In some embodiments, different 5′-nuclease fluorescent probes, each fluorescing at different wavelengths, can distinguish between different amplification products within the same amplification reaction. For example, in some embodiments, one could use two different 5′-nuclease fluorescent probes that fluoresce at two different wavelengths (WL_{A} and WL_{B}) and that are specific to two different stem regions of two different extension reaction products (A′ and B′, respectively). Amplification product A′ is formed if target nucleic acid sequence A is in the sample, and amplification product B′ is formed if target nucleic acid sequence B is in the sample. In some embodiments, amplification product A′ and/or B′ may form even if the appropriate target nucleic acid sequence is not in the sample, but such occurs to a measurably lesser extent than when the appropriate target nucleic acid sequence is in the sample. After amplification, one can determine which specific target nucleic acid sequences are present in the sample based on the wavelength of signal detected and their intensity. Thus, if an appropriate detectable signal value of only wavelength WL_{A} is detected, one would know that the test sample includes target nucleic acid sequence A, but not target nucleic acid sequence B. If an appropriate detectable signal value of both wavelengths WL_{A} and WL_{B} are detected, one would know that the test sample includes both target nucleic acid sequence A and target nucleic acid sequence B. In some embodiments, detection can occur through any of a variety of mobility dependent analytical techniques based on differential rates of migration between different analyte species. Exemplary mobility-dependent analysis techniques include electrophoresis, chromatography, mass spectroscopy, sedimentation, e.g., gradient centrifugation, field-flow fractionation, multi-stage extraction techniques, and the like. In some embodiments, mobility probes can be hybridized to amplification products, and the identity of the target polynucleotide determined via a mobility dependent analysis technique of the eluted mobility probes, as described for example in Published P.C.T. Application WO04/46344 to Rosenblum et al., and WO01/92579 to Wenz et al., In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003). It will also be appreciated that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. Detection of unlabeled reaction products, for example using mass spectrometry, is also within the scope of the present teachings.

As used herein the term “derived micro RNA quantity” refers to a quantity for a micro RNA that results for an experiment performed on a test sample, according to the methods of the present teachings. The derived micro RNA quantity can be calculated by comparison to an endogenous control small nucleic acid. Once the derived micro RNA quantity is determined, it can be compared to the expectation micro RNA quantity, and the diagnosis of a biological condition performed.

As used herein, the term “abundantly expressed” refers to an RNA molecule, typically a micro RNA, which is expressed at typically several thousand copies per cell. The term “minimally expressed” refers to an RNA molecule, typically a micro RNA, which is expressed at fifty or fewer copies per cell.

### Exemplary Embodiments

Assays for Quantifying Micro RNAs as Biomarkers

In a first aspect, the present teachings provide assay methods for the amplification and quantitation of one or a plurality of micro RNA molecules that are known biomarkers for a tissue of interest, wherein the micro RNAs are expressed in a test sample. Often, the test sample is recovered from one or more background tissues that differ from the tissue of interest, both in normal location as well as in micro RNA biomarker profile.

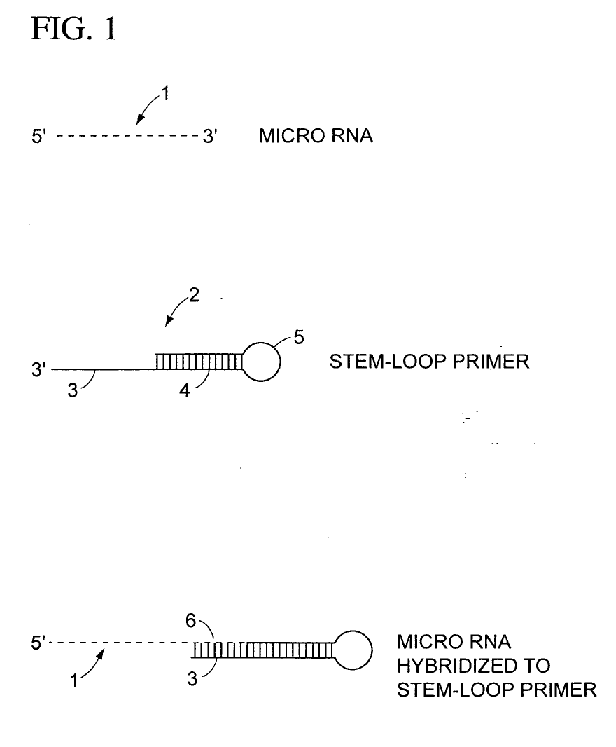

FIG. 1 depicts certain compositions according to some embodiments of the present teachings. Top, a miRNA molecule (1, dashed line) is depicted. Middle, a stem-loop primer (2) is depicted, illustrating a 3′ target specific portion (3), a stem (4), and a loop (5). Bottom, a miRNA hybridized to a stem-loop primer is depicted, illustrating the 3′ target specific portion of the stem-loop primer (3) hybridized to the 3′ end region of the miRNA (6).

As shown in FIG. 2, a target polynucleotide (9, dotted line) is illustrated to show the relationship with various components of the stem-loop primer (10), the detector probe (7), and the reverse primer (8), according to various non-limiting embodiments of the present teachings. For example as shown in FIG. 2A, in some embodiments the detector probe (7) can correspond with the 3′ end region of the target polynucleotide in the amplification product as well as a region upstream from the 3′ end region of the target polynucleotide in the amplification product. (Here, the detector probe is depicted as rectangle (7) with an F and a Q, symbolizing a TaqMan probe with a florophore (F) and a quencher (Q)). Also shown in FIG. 2A, the loop can correspond to the reverse primer (8). In some embodiments as shown in FIG. 2B, the detector probe (7) can correspond with a region of the amplification product corresponding with the 3′ end region of the target polynucleotide in the amplification product, as well as a region upstream from the 3′ end region of the target polynucleotide in the amplification product, as well as the stem-loop primer stem in the amplification product. Also shown in FIG. 2B, the upstream region of the stem, as well as the loop, can correspond to the reverse primer (8). In some embodiments as shown in FIG. 2C, the detector probe can correspond to the amplification product in a manner similar to that shown in FIG. 2B, but the loop can correspond to the reverse primer (8). In some embodiments as shown in FIG. 2D, the detector probe (7) can correspond with the stem-loop primer stem in the amplification product. Also shown in FIG. 2D, the upstream region of the stem, as well as the loop can correspond to the reverse primer (8). It will be appreciated that various related strategies for implementing the different functional regions of these compositions are possible in light of the present teachings, and that such derivations are routine to one having ordinary skill in the art without undue experimentation.

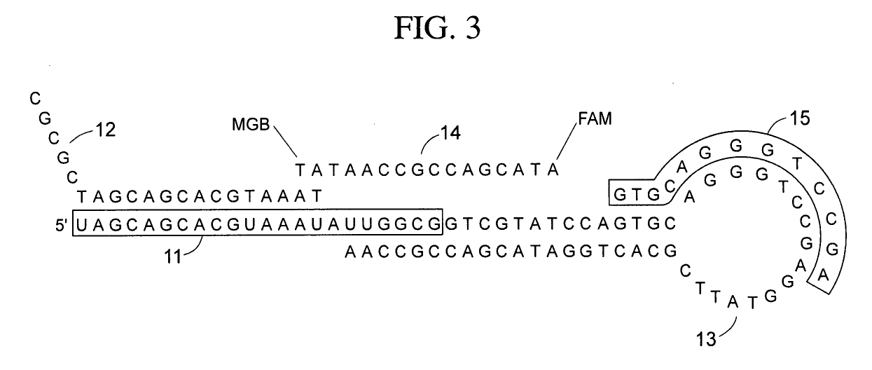

FIG. 3 depicts the nucleotide relationship for the micro RNA MiR-16 (boxed, 11) according to some embodiments of the present teachings. Shown here is the interrelationship of MiR-16 to a forward primer (12), a stem-loop primer (13), a TaqMan detector probe (14), and a reverse primer (boxed, 15). The TaqMan probe comprises a 3′ minor groove binder (MGB), and a 5′ FAM florophore. It will be appreciated that in some embodiments of the present teachings the detector probes, such as for example TaqMan probes, can hybridize to either strand of an amplification product. For example, in some embodiments the detector probe can hybridize to the strand of the amplification product corresponding to the first strand synthesized. In some embodiments, the detector probe can hybridize to the strand of the amplification product corresponding to the second strand synthesized. Thus, the sequences presented in FIG. 3 include:

| | |
|---|---|
| SEQ ID NO: 4 | 5′CGCGCTAGCAGCACGTAAAT3′ |
| SEQ ID NO: 5 | 5′6-FAM-ATACGACCGCCAATAT-MGB3′ |
| SEQ ID NO: 6 | 5′AGCCTGGGACGTG3′ |
| SEQ ID NO: 7 | 5′AACCGCCAGCATAGGTCACGCTTATGGAGCCTGGG |
| | ACGTGACCTATGCTG3′ |
| SEQ ID NO: 8 | 5′UAGCAGCACGUAAAUAUUGGCG3′ |

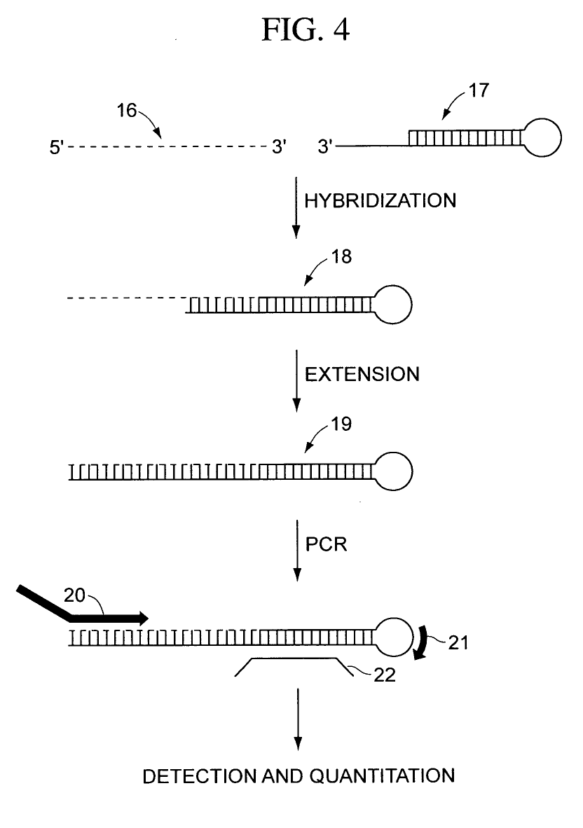

FIG. 4 depicts a single-plex assay design according to some embodiments of the present teachings. Here, a miRNA molecule (16) and a stem-loop primer (17) are hybridized together (18). The 3′ end of the stem-loop primer of the target-stem-loop primer composition is extended to form an extension product (19) that can be amplified in a PCR. The PCR can comprise a miRNA specific forward primer (20) and a reverse primer (21). The detection of a detector probe (22) during the amplification allows for quantitation of the miRNA.

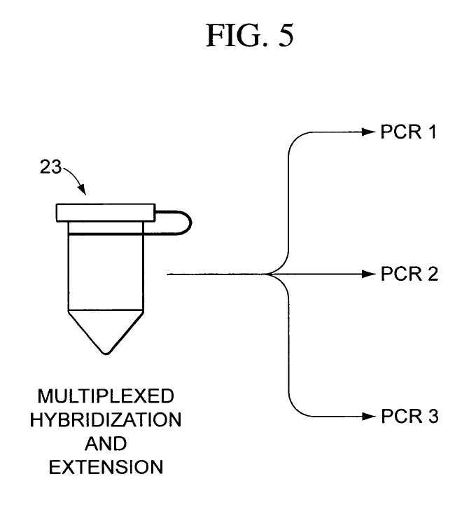

FIG. 5 depicts an overview of a multiplex assay design according to some embodiments of the present teachings. Here, a multiplexed hybridization and extension reaction is performed in a first reaction vessel (23). Thereafter, aliquots of the extension reaction products from the first reaction vessel are transferred into a plurality of amplification reactions (here, depicted as PCRs 1, 2, and 3) in a plurality of second reaction vessels. Each PCR can comprise a distinct primer pair and a distinct detector probe. In some embodiments, a distinct primer pair but the same detector probe can be present in each of a plurality of PCRs.

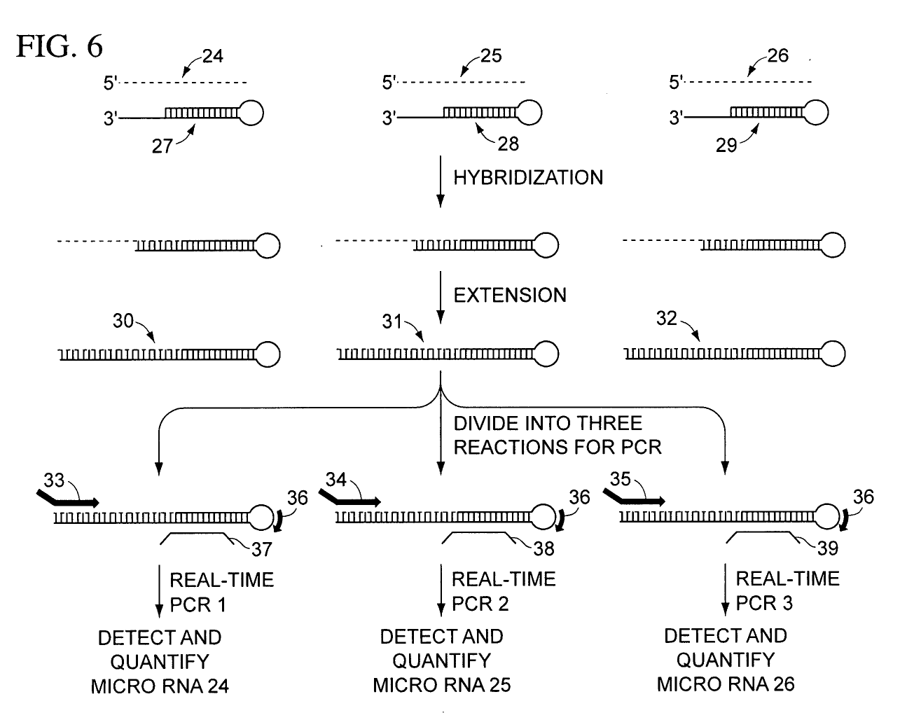

FIG. 6 depicts a multiplex assay design according to some embodiments of the present teachings. Here, three different miRNAs (24, 25, and 26) are queried in a hybridization reaction comprising three different stem-loop primers (27, 28, and 29). Following hybridization and extension to form extension products (30, 31, and 32), the extension products are divided into three separate amplification reactions. (Though not explicitly shown, it will be appreciated that a number of copies of the molecules depicted by 30, 31, and 32 can be present, such that each of the three amplification reactions can have copies of 30, 31, and 32.) PCR 1 comprises a forward primer specific for miRNA 24 (33), PCR 2 comprises a forward primer specific for miRNA 25 (34), and PCR 3 comprises a forward primer specific for miRNA 26 (35). Each of the forward primers further comprises a non-complementary tail portion. PCR 1, PCR 2, and PCR 3 all comprise the same universal reverse primer 36. Further, PCR 1 comprises a distinct detector probe (37) that corresponds to the 3′ end region of miRNA 24 and the stem of stem-loop primer 27, PCR 2 comprises a distinct detector probe (38) that corresponds to the 3′ end region of miRNA 25 and the stem of stem-loop primer 28, and PCR 3 comprises a distinct detector probe (39) that corresponds to the 3′ region of miRNA 26 and the stem of stem-loop primer 29.

Additional description of approaches for amplifying and quantifying micro RNAs using stem-loop primers can be found in U.S. patent application Ser. No. 10/947,460 to Chen et al.,. Various multiplexed approaches that can be used in the context of the present teachings are further described in co-filed U.S. Non-Provisional Patent Application Multiplexed Amplification of Short Nucleic Acids, claiming priority to U.S. Provisional Application 60/686,521 filed May 31, 2005, and to U.S. Provisional Application 60/708,946, filed Aug. 16, 2005, and to U.S. Provisional Application 60/711,480, filed Aug. 24, 2005, and to U.S. Provisional Application 60/781,208, filed Mar. 10, 2006, and to U.S. Provisional Application 60/790,472, filed Apr. 7, 2006, and to U.S. Provisional Application Methods for Characterizing Cells Using Amplified Micro RNAs filed May 15, 2006.

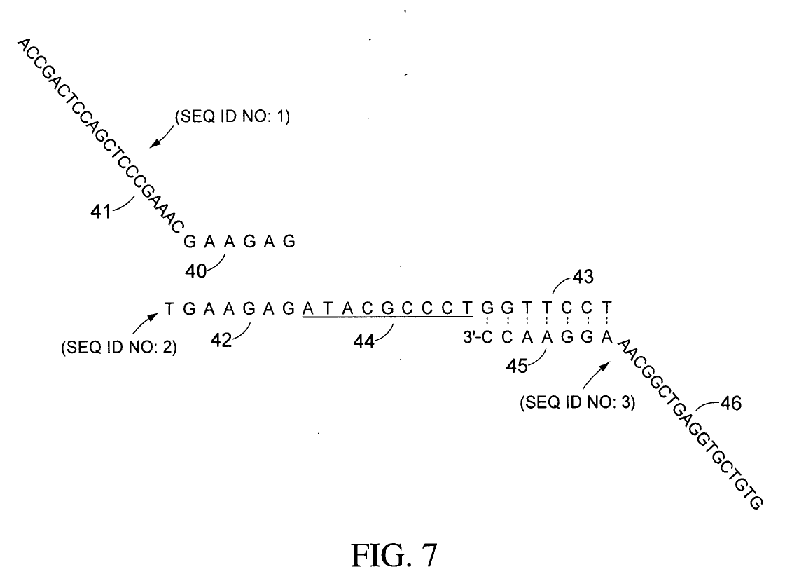

As another example of an assay design according to some embodiments of the present teachings, FIG. 7 depicts a first primer (SEQ ID NO:1) of an illustrative first primer set that includes a target-binding portion (40) and a second portion (41) that is upstream from the target-binding portion (40); a polynucleotide target (SEQ ID NO:2) that includes a first target region (42) a second target region (43), and in this example, a stretch of gap sequences (44; shown underlined); and a corresponding reverse primer (SEQ ID NO:3) of the illustrative first primer set that includes a target-binding portion (45) and a second portion (46) that is upstream from target-binding portion (45). Additional description of approaches for amplifying and quantifying micro RNAs using PCR approaches can be found in U.S. patent application Ser. No. 10/944,153 to Lao et al., Accompanying sequences for these reagents are:

| | |
|---|---|
| SEQ ID NO 1: | 5′ACCGACTCCAGCTCCCGAAACGAAGAG3′ |
| SEQ ID NO 2: | 5′TGAAGAGATACGCCCTGGTTCCT3′ |
| SEQ ID NO 3: | 5′GTGTCGTGGAGTCGGCAAAGGAACC3′ |

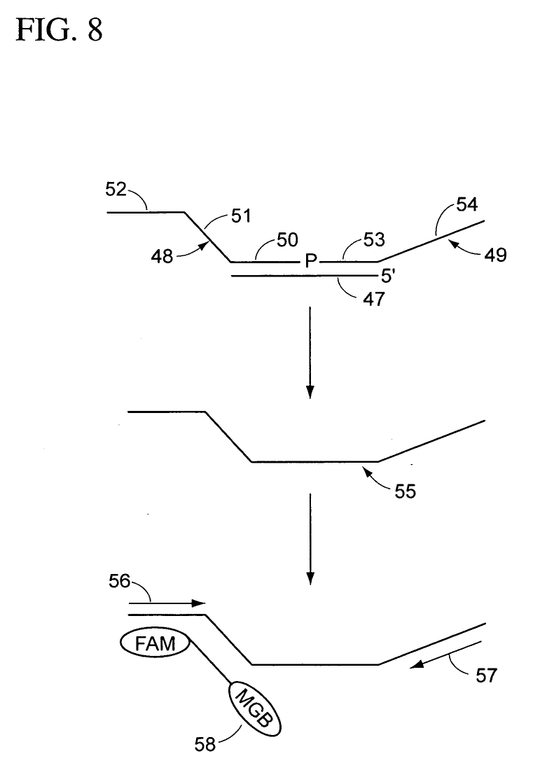

As another example of an assay design according to some embodiments of the present teachings, FIG. 8 depicts a target micro RNA (47) being queried in a ligation reaction comprising a first ligation probe (48) and a second ligation probe (49). The first ligation probe can comprise a target specific portion (50), a target identifying portion (51) and a forward primer portion (52). The second ligation probe can comprise a 5′ phosphate group (P), a target specific portion (53) and a reverse primer portion (54). The resulting ligation product (55) can be amplified in a PCR with a forward primer (56) and a reverse primer (57), wherein a detector probe such as a TaqMan® probe (58, shown with a FAM label and a minor groove binder (MGB)) hybridizes to the identifying portion, or identifying portion complement, that was introduced into the ligation product by the first ligation probe. Additional description of approaches for amplifying and quantifying micro RNAs using ligation probes comprising identifying portions can be found in U.S. patent application Ser. No. 10/881,362 to Brandis et al.

The methods provided in FIGS. 1-9 can be applied in a variety of assay configurations according to the present teachings. For example, a multiplexed reverse transcription reaction can be performed with a plurality of micro RNA specific stem-loop primers. The reverse transcription reaction can then be divided (split) into a plurality of PCR amplification reactions, wherein each PCR comprises a micro RNA specific forward primer, a universal reverse primer, and a micro RNA specific detector probe. Additional illustrations such approaches can be found in U.S. patent application Ser. No. 10/947,460 to Chen et al., co-filed U.S. Patent Application Methods for Characterizing Cells Using Amplified Micro RNAs claiming priority to U.S. Provisional Application 60/686,521 and 60/708,949, and co-filed U.S. Patent Application Multiplexed Amplification of Short Nucleic Acids, claiming a priority to U.S. Provisional Patent Application No. 60/686,521, filed May 31, 2005, U.S. Patent Provisional Application No. 60/708,946, filed Aug. 16, 2005, U.S. Provisional Patent Application No. 60/711,480, filed Aug. 24, 2005, U.S. Provisional Patent Application No. 60/781,208, filed Mar. 10, 2006, U.S. Provisional Patent Application No. 60/790,472, filed Apr. 7, 2006, and U.S. Provisional Patent Application No. 60/800,376, filed May 15, 2006.

In another example of the assay configurations contemplated by the present teachings, a multiplexed cycling reverse transcription can be performed with a plurality of micro RNA specific stem-loop primers to provide a linear amplification of the micro RNAs. Following the multiplexed cycling reverse transcription, the amplified products can be split into a plurality of PCR amplification reactions, wherein each PCR comprises a micro RNA specific forward primer, a universal reverse primer, and a micro RNA specific detector probe. Additional illustrations of such cycling reverse transcription approaches can be found in the co-filed U.S. Non-Provisional Application Linear Amplification of Short Nucleic Acids to Bloch, claiming priority to U.S. Provisional Application 60/789,752.

Additional approaches to performing multiplexed amplification reactions within the scope of the present teachings can be found in the co-filed application U.S. Non-Provisional Patent Application Methods for Characterizing Cells Using Amplified Micro RNAs claiming priority to U.S. Provisional Application 60/686,521 and 60/708,946 which describes multiplexed cycling reverse transcription reactions coupled with multiplexed PCR pre-amplification reactions. Additional teachings regarding multiplexed PCR pre-amplification reactions can be found in U.S. Pat. No. 6,605,451 to Xtrana. Various encoding/decoding reaction schemes discussed in U.S. patent application Ser. No. 11/090,468 to Lao et al., and U.S. patent application Ser. No. 11/090,830 to Andersen et al., can also be applied in the present teachings.

The methods and kits of the present teachings provide for increased levels of sensitivity, dynamic range, and throughput in quantifying micro RNAs in various diagnostic, research, and applied settings. The exponential PCR amplification, for example, can provide sensitivity of detection down to potentially a single molecule of micro RNA.

In some embodiments, sensitivity of detection of less than 5 molecules of micro RNA is contemplated. In some embodiments, sensitivity of detection of less than 10 molecules of micro RNA is contemplated. In some embodiments, sensitivity of detection of less than 50 molecules of micro RNA is contemplated. Further, real-time PCR as employed in the present teachings can provide for an enormous dynamic range, enabling the quantitation of expression levels ranging up to 9 orders of magnitude. In some embodiments, quantitation of expression levels ranging up to 8 orders of magnitude is contemplated. In some embodiments, quantitation of expression levels ranging up to 7 orders of magnitude is contemplated. In some embodiments, quantitation of expression levels ranging up to 6 orders of magnitude is contemplated. In some embodiments, quantitation of expression levels ranging up to 5 orders of magnitude is contemplated. In some embodiments, quantitation of expression levels ranging up to 4 orders of magnitude is contemplated. It will be appreciated, and discussed further elsewhere in the present teachings, that the assays of the present teachings can be applied in contexts in which a single target micro RNA is queried, as well as contexts in which a plurality of different target micro RNAs are queried.

Methods of Diagnosing Biological Conditions, Including Cancer

In a second aspect, the present teachings provide methods and biomarkers for determining a biological condition, including for example cellular identification and disease diagnosis, especially in the context of cancer. The present teachings can be employed on test samples comprising very small numbers of cells.

Historically, one commonly used approach to cellular identification employs analysis of messenger RNA (mRNA). Despite years of efforts devoted to developing robust mRNA biomarkers for metastatic cancer there currently are no mRNA biomarkers that can be assayed with sufficient accuracy and sensitivity to allow scarce micrometastases of the most aggressive cancers (breast, prostate, colon, lung) to be identified reliably in background tissue such as blood, bone marrow, lymph node, and/or other solid tissues.

The present teachings enable cellular identification, in the context of cancer diagnosis and any number of other areas, by providing assays for the quantitative analysis of target micro RNA s. By assaying and quantifying the presence of target micro RNA s present ectopically in one or more background tissues, the present teachings address, for example, the problematic issue of identifying the primary tumor responsible for clinically identified metastatic foci.

Cancer cell detection has historically been hampered by the considerable biochemical diversity (for example, messenger RNA) present in neoplasia, resulting in unacceptable false positive and false negative results. Such biochemical diversity in messenger RNA expression is problematic both in the attempt to infer the presence of disease from the presence of tissue-specific biomarkers, as well as in the attempt to infer the presence of disease from the presence of disease-specific biomarkers. For example, the use of tissue-specific biomarkers to infer the presence of metastatic disease from the presence of epithelial cells in non-epithelial background tissue such as bone marrow has suffered from unacceptable levels of sensitivity and specificity (see for example Lambrechts et al., Breast Cancer Research Tr. 56: 219-231). Messenger RNA biomarkers are especially difficult to quantitate in histological cell preparations (for example, fixed, stained, tissue mounted on a microscope slide), simply because messenger RNA is degraded by the ubiquitous and difficult to denature ribonucleases that can contaminate these biological samples.

In contrast to messenger RNA, micro RNAs are bound and protected by specific intracellular proteins, forming protein-RNA complexes known as miRNP (see Mourelatos et al., (2002), Genes and Development 16:720-728). Informative messenger RNA biomarker profiles (for example, for use as clinically effective tissue biomarkers) are likely to comprise hundreds of distinct sequences, thus creating a technical problem separating the diagnostic signal from background signals as well as the economic problem of developing cost-effective diagnostic assays. In contrast, higher organisms possess the genes for only about 200 distinct micro RNAs, only a small subset of which should suffice for distinguishing the tissue of interest from background tissue(s).

According to the present teachings, the test sample can undergo any of a variety of sample preparation procedures known in the art to prepare nucleic acid molecules for analysis. For example, in some embodiments of the present teachings, the test sample undergoes a heat lysis treatment, and micro RNA quantified thereafter. In some embodiments, especially when the test sample is blood, the test sample can be collected in a commercially available Tempus Tube™ from Applied Biosystems, and micro RNA quantified thereafter. In some embodiments, various other sample preparation procedures commonly employed in the art of molecular biology can be employed, including for example the mirVana micro RNA isolation kit (commercially available from Ambion) and the 6100 nucleic acid sample prep products commercially available from Applied Biosystems, as well as various lysis approaches discussed in U.S. Non-Provisional patent application Ser. No. 10/947,460 to Chen et al.

In some embodiments, cells to be analyzed according to the present teachings can be collected in a manner similar to that employed for the collection of platelets in platelet donors. For example, extracting platelet cells from a donor's body uses a cell separation machine. The blood flows from the donor into the cell separation machine and the blood components can be separated into different layers by centrifugation. A local anaesthetic agent can be given before inserting each needle into the donor's arms. Each needle is connected to the cell separation machine and blood is drawn from one arm. Separation techniques, for example differential centrifugation can then be employed to separate ectopic circulating cells of interest. Additional such approaches for collecting circulating cancer cells can be found in Cristofanilli et al., Journal of Clinical Oncology, 23:7, Mar. 1, 2005. In some embodiments, it is possible that the test sample contains no background tissue, for example if 100 percent purity of cells of interest are obtained from the test sample.

In some embodiments, the present teachings can be applied in the context of cancer diagnosis. For example, the tissue of interest is epithelial tissue (epithelium from an organ) and can be from an organ that can give rise to metastatic cancer, such as breast, prostate, colon, skin, or lung. In such a context, a cell mass can be a metastasis, and the background tissue comprises any other organ, such as blood, cerebrospinal fluid, saliva, or excreta such as stool, urine, or mucus. A test sample collected from any these (or other) background tissues could potentially contain cancerous epithelial cells in addition to the background tissue(s). A panel of target micro RNA s (a “signature”) expressed in epithelial cells can be quantitated according to the assays of the present teachings to infer the diagnosing a biological condition, and hence whether the test sample comprises cancerous epithelial cells. In some embodiments, target micro RNA s expressed in background tissues can also be quantitated according to the assays of the present teachings.

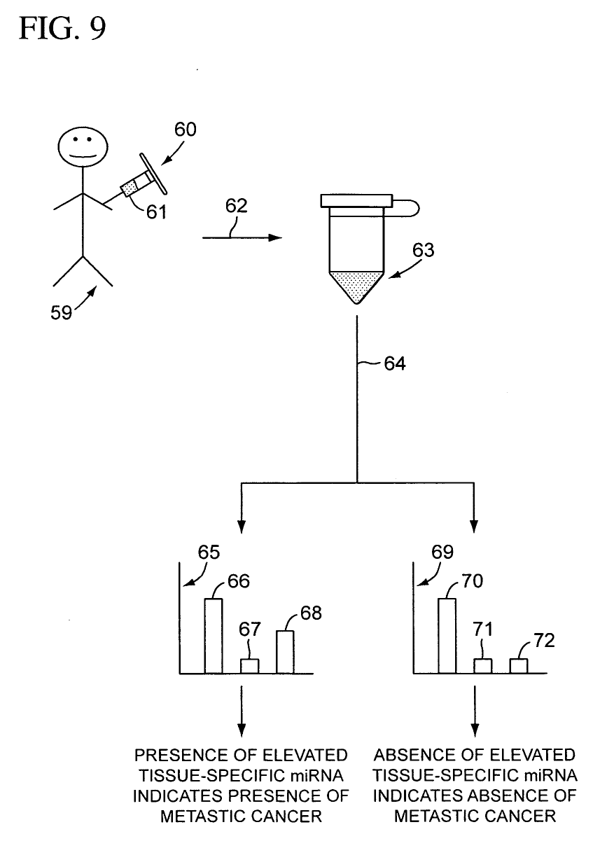

For example in FIG. 9, a patient (59) is depicted, in which a test sample (61, here blood) is collected from the patient's arm using a syringe (60). Following an appropriate sample preparation procedure (62) such as heat lysing, or the use of commercially available Applied Biosystems Tempus Tubes™, the prepared test sample (63) is subjected to an assay (64) according to the methods of the present teachings, for example a commercially available real time PCR assay employing micro RNA specific stem-loop primers and TaqMan™ detector probes (Applied Biosystems TaqMan® Micro RNA Assays), along with an endogenous control small RNA (see infra regarding “Controls”). There are two possible resulting graphs (65 or 69), depending on the nature of the biological condition to be diagnosed. The Y way axis of each graph indicates quantity of a target micro RNA. All the bars refer to quantities of a single hypothetical target micro RNA. Graph 65 indicates an abundant expectation micro RNA quantity in the tissue of interest (bar 66), a minimal expectation micro RNA quantity in the background tissue (bar 67), and an intermediate derived micro RNA quantity resulting from the experiment (bar 68). Thus, graph 65 indicates the presence of an elevated tissue-specific micro RNA in the test sample, and thus can indicate the presence of metastatic cancer. Graph 69 on the other hand illustrates the result of an experiment on an analogous test sample, taken from a different clinical patient, which indicates the absence of metastatic cancer. Specifically, bar 70 indicates an abundant expectation micro RNA quantity in the tissue of interest, bar 71 indicates a minimal expectation micro RNA quantity in the background tissue, and bar 72 indicates a minimal derived micro RNA quantity in the test sample. Thus, graph 69 indicates the absence of an elevated tissue-specific micro RNA in the test sample, and thus can indicate the absence of metastatic cancer. Not shown in FIG. 9, but also contemplated by the present teachings, and elaborated on below under Controls, is the comparison of the derived micro RNA quantities to endogenous control small nucleic acids. The depicted graphs shown in FIG. 9 presume that such normalization has occurred. Such endogenous controls can have the function of normalizing the derived micro RNA quantity for such potentially confounding variables as differences in sample input and differences in reaction efficiency.

Usually, diagnostic decisions will be made not on single target micro RNA quantities, but rather a signature of micro RNA quantities, wherein statistical analysis confirms that the profile is atypical of background tissue, and can be explained by admixture of some number of cells, possibly quite small, from a tissue of interest. Such procedures are referred to in the art as “expression profiling,” and are discussed for example in Nature Genetics, The Chipping Forecast (June 2005) volume 37, s6.

Without intending to be limiting, a number of representative examples for cancer detection enabled by the present teachings can be inferred from tissue distribution studies of micro RNA sequences (see for example published PCT Application US/2003/041549). Such examples include the detection of increased mir-15 micro RNA in a test sample collected from a tissue site other than prostate, and inferring therefrom an increased likelihood that the diagnosing a biological condition indicative of prostate cancer. Detection of increased mir-35 micro RNA in a test sample collected from a tissue site other than kidney, and inferring therefrom an increased likelihood that the biological condition is indicative of kidney cancer. Detection of increased mir-16 micro RNA in a tissue site other than brain, kidney, liver, and lung, and inferring therefrom an increased likelihood that the biological condition is indicative of cancer in any one of brain, liver, and lung. Other studies indicate, for example, that detection of mir-375 in a test sample collected from a tissue site other than pancreatic islet cells can be indicative of pancreatic cancer (see for example, Poy et al., Nature, (2004) Nov. 11; 432(7014):226-30). TaqMan® assays for these and numerous other micro RNAs are commercially available from Applied Biosystems.

In the context of cancer diagnosis and other application areas, the present teachings further contemplate embodiments in which small numbers of cells are analyzed (also see co-filed U.S. Non-Provisional Patent Application Methods for Characterizing Cells Using Amplified Micro RNAs claiming priority to U.S. Provisional Patent Application 60/686,521, and 60/708,946. In some embodiments, the present teachings provide for analysis of one or more target micro RNA molecules in a single cell. In some embodiments, the present teachings provide for analysis of one or more target micro RNA molecules in five or fewer cells. In some embodiments, the present teachings provide for analysis of one or more target micro RNA molecules in ten or fewer cells. In some embodiments, the present teachings provide for analysis of one or more target micro RNA molecules in fifty or fewer cells. In some embodiments, the present teachings provide for analysis of one or more target micro RNA molecules in one hundred and fifty or fewer cells. In some embodiments, the present teachings provide for analysis of one or more target micro RNA molecules in greater than one hundred and fifty cells. As discussed supra, any of a variety of amplification strategies can be employed in the context of the present teachings for the analysis of small numbers of cells. The test samples from which such small numbers of cells can be recovered comprise conventionally fixed and stained histological and cytological preparations on microscope slides, single cells dissected from early-stage embryos generated by in vitro fertilization, microdissected needle-biopsy cores, blood samples, and forensics samples. Laser-capture microdissection is another attractive method of recovering diagnostic cells from histological preparations. Such laser-capture systems are commercially available from such sources as Arcturus (for example, the Veritas™ Microdissection Instrument).

In some embodiments, therapies can be designed based on the miRNAs and mRNAs that are differentially expressed, using for example the tools of siRNA and RNAi, as well as antagomirs (Krutzfeldt et al., 2005 Dec. 1; 438(7068):685-9).

While FIG. 9 as depicted and described illustrates some embodiments of the present teachings in the context of cancer diagnosis, it will be appreciated that the present teachings can be applied in any number of contexts in which a target micro RNA is quantified in test sample comprising at least one of a background tissue and, potentially, a tissue of interest, including for example the examination of stem cells, as further in co-filed U.S. Non-Provisional Patent Application Methods for Characterizing Cells Usinq Amplified Micro RNAs claiming priority to U.S. Provisional Patent Application 60/686,521, and 60/708,947.

Controls

In a third aspect, the present teachings contemplate embodiments in which a co-assay is performed in parallel with the one or more target micro RNA s, wherein the amplification reaction further comprises specific short RNA sequences that are present intracellularly in small ribonucleoproteins (snRNP) with ‘housekeeping’ functions. Such snRNPs can serve as quantitative normalization controls as endogenous control small RNAs. For example, endogenous control RNAs can include U7, U8, U11, U13, U3, U12, and others (see for example Basenga and Steitz, pp. 359-381 in Gesteland and Atkins (1993) The RNA World, Cold Spring Harbor Press, and Yu et al., pp. 487-524 in Gesteland et al., (1999) The RNA World, Cold Spring Harbor Press. In some embodiments, the endogenous controls are expressed in cells at a level of about 5000-40,000 copies per cell, relatively independent of cell type. This kind of quantitative range is comparable to that of highly expressed micro RNA and therefore is unlikely to stoichiometrically overwhelm the amplification reaction component of the assay. In some embodiments, controls nucleic acids are chosen that comprise expression levels of 10³-10⁴ molecules per cell.

In some embodiments the endogenous control small RNAs can include U7, U8, U11, U13, U3, and U12. The primers querying these endogenous control small RNAs are designed to query single stranded regions, such single stranded regions comprising about 16 to about 36 nucleotides in length, thereby avoiding potential accessibility problems presented by such snRNP as U3 and U12, which themselves comprise single-stranded regions of only around 9-14 nucleotides. Querying single-stranded regions 16-36 nucleotides in length can obviate the accessibility problems of U3 and U12. Thus, in some embodiments, the single stranded regions are at least 18 nucleotides in length.

In some embodiments of the present teachings, micro RNA expression levels can be normalized to the number of cells directly measured in the test sample by conventional means. In some embodiments of the present teachings, it can be easier and cheaper to normalize to the expression levels measured for housekeeping small RNA in parallel to those for the target micro RNA, which in turn can be calibrated on a per-cell basis in separate reactions. For certain samples, such as for example solid tumor lumps and needle biopsies, direct cell counting is especially difficult, and thus normalizing the quantity of target micro RNA in a parallel amplification reaction can be desirable. In some embodiments, the endogenous control sequence can be a micro RNA that is normally abundantly expressed in background tissue, and minimally expressed in the tissue of interest.

In some embodiments, the quantity of the endogenous control small RNA correlates negatively with the quantity of the target micro RNA when the tissue of interest and the background tissue are compared to one another, thus enhancing sensitivity when the test sample comprises cells from the tissue of interest.

In some embodiments, the quantity of the target micro RNA is normalized to a measure of background cell number found in a test aliquot derived from the test sample.

In some embodiments, the quantity of the target micro RNA is normalized to a quantity of an endogenous control small RNA in a test aliquot derived from the test sample

In some embodiments, the endogenous control small RNA is a micro RNA expressed abundantly in the background tissue.

In some embodiments, the endogenous control small RNA is amplified in the same reaction mixture as the target micro RNA.

In some embodiments, the endogenous control small RNA is selected from the group consisting of U7, U8, U11, U13, U3, and U12.

In some embodiments, the endogenous control small RNA is abundantly expressed in the background tissue and the target micro RNA is minimally expressed in the tissue of interest.

In some embodiments, the endogenous control small RNA is abundantly expressed in the background tissue and the target micro RNA is abundantly expressed in the tissue of interest.

In some embodiments, a single stranded region of the endogenous control RNA is queried in the amplification reaction, wherein the single stranded region is chosen based on a secondary structure prediction of the endogenous control small RNA, and wherein the secondary structure prediction indicates the presence of a single stranded region that is at least 18 nucleotides in length.

In some embodiments, the expectation micro RNA quantity has been established in advance of the amplification reaction through calibration of micro RNA expression in reference tissue samples. For example, the quantity of a target micro RNA in a known number of cells in a tissue of interest such as prostate can be known from previous studies, and stored in the software that analyzes the production of the derived micro RNA quantity. When a test sample undergoes amplification according to the methods of the present teachings, and the test sample comprises a derived micro RNA quantity that exceeds the value of the expected target micro RNA quantity stored in the software, the biological condition of prostate cancer is thereby diagnosed.

In some embodiments, the expectation micro RNA quantity is established by simultaneous parallel analysis of micro RNA expression in test sample and one or more reference tissue samples.

Additional embodiments discussing how endogenous controls can be employed in the context of the present teachings can be found in U.S. Non-Provisional Application Endogenous Controls for Quantifying Micro RNAs, claiming priority to U.S. Provisional Application 60/686,274 and 60/670,790.

While the present teachings have been described in terms of these exemplary embodiments, the skilled artisan will readily understand that numerous variations and modifications of these exemplary embodiments are possible without undue experimentation. All such variations and modifications are within the scope of the present teachings. Aspects of the present teachings may be further understood in light of the following claims.

We claim:

1. A method for diagnosing a biological condition comprising;
amplifying a target micro RNA from a test sample to provide a derived micro RNA quantity, wherein the target micro RNA is from a tissue of interest;
comparing the derived micro RNA quantity to an expectation micro RNA quantity from a background tissue; and,
diagnosing the biological condition.

2. The method according to claim 1 wherein the amplifying is an exponential amplification reaction of a target micro RNA.

3. The method according to claim 1 wherein the amplifying is a linear amplification reaction.

4. The method according to claim 1 wherein the background tissue is at least one of blood, a cellular sub-fraction of blood, lymph, lymph node, spleen, bone marrow, bone, cerebrospinal fluid, any solid cell mass suspected of comprising metastatic cancer cells, or combinations thereof.

5. The method according to claim 1 wherein the tissue of interest is lung, breast, prostate, cervical epithelium, skin, B-lymphocytes, T-lymphocytes, granulocytes, or colon epithelium.

6. The method according to claim 1 wherein the test sample is a core from a needle biopsy, an aspirate from a needle biopsy, a dissected sub-fraction of a surgically removed cell mass, histochemically identified sub-fraction of a tissue section, histochemically identified sub-fraction of a cytospin preparation, at least one cell isolated by laser capture microdissection, intravenous blood draw, finger prick, a subfraction of a cell suspension subjected to MACS, a subfraction of cell suspension subjected to a FACS, a subfraction of cell suspension subjected to an immunoprecipitation, or a subfraction of a cell suspension subjected to density centrifugation.

7. The method according to claim 1 wherein the target micro RNA to be amplified is present in no more than 150 copies in the test sample.

8. The method according to claim 1 wherein the target micro RNA to be amplified is present in no more than 75 copies in the test sample.

9. The method according to claim 1 wherein the target micro RNA to be amplified is present in no more than 25 copies in the test sample.

10. The method according to claim 1 wherein the target micro RNA to be amplified is present in no more than 5 copies in the test sample.

11. The method according to claim 2 wherein the dynamic range of the amplifying is not less than three powers of ten.

12. The method according to claim 2 wherein the dynamic range of the amplifying is not less than four powers of ten.

13. The method according to claim 2 wherein the dynamic range of the amplifying is not less than five powers of ten.

14. The method according to claim 2 wherein the dynamic range of the amplifying is not less than six powers of ten.

15. The method according to claim 1 wherein the quantity of the target micro RNA is normalized to a measure of background cell number found in a test aliquot derived from the test sample.

16. The method according to claim 1 wherein the quantity of the target micro RNA is normalized to a quantity of an endogenous control small RNA in a test aliquot derived from the test sample

17. The method according to claim 16 wherein the endogenous control small RNA is expressed abundantly in the background tissue.

18. The method according to claim 17 wherein the endogenous control small RNA is amplified in the same reaction mixture as the target micro RNA.

19. The method according to claim 16 wherein the endogenous control small RNA is selected from the group consisting of U7, U8, U11, U13, U3, and U12.

20. The method according to claim 16 wherein the endogenous control small RNA is abundantly expressed in the background tissue, and the target micro RNA is minimally expressed in the tissue of interest.

21. The method according to claim 16 wherein the endogenous control small RNA is abundantly expressed in the background tissue and the target micro RNA is abundantly expressed in the tissue of interest.

22. The method according to claim 19 wherein a single stranded region of the endogenous control small RNA is queried in the amplification reaction, wherein the single stranded region is chosen based on a secondary structure prediction of the endogenous control small RNA, and wherein the secondary structure prediction indicates the presence of a single stranded region that is at least 18 nucleotides in length.

23. The method according to claim 1 wherein the expectation micro RNA quantity has been established in advance of the amplification reaction through calibration of micro RNA expression in reference tissue samples.

24. The method according to claim 1 wherein the expectation micro RNA quantity is established by simultaneous parallel analysis of micro RNA expression in test sample and one or more reference tissue samples.

25. The method according to claim 2 wherein the exponential amplification reaction comprises reverse transcription-polymerase chain reaction (RT-PCR).

26. The method according to claim 25 wherein the RT-PCR comprises a real-time read-out.

27. The method according to claim 26 wherein the real-time read-out comprises a real-time probe, wherein the real time probe is selected from the group consisting of a DNA-binding dye, a TaqMan® probe, a molecular beacon, and a PNA probe.

28. The method according to claim 2 wherein the exponential amplification reaction comprises extension of a stem-loop primer hybridized to the target micro RNA followed by a PCR, wherein a reverse primer in the PCR corresponds to a loop region of the stem-loop primer, wherein a forward primer in the PCR comprises an extension reaction product portion and a tail portion, and wherein the PCR comprises a detector probe, wherein the detector probe comprises sequence corresponding to a stem of the stem-loop primer and the target micro RNA.

29. The method according to claim 28 wherein the tissue of interest is prostate, the test sample comprises blood, the biological condition is prostate cancer, and the target micro RNA is mir-15.

30. The method according to claim 28 wherein the tissue of interest is kidney, the test sample comprises blood, the biological condition is kidney cancer, and the target micro RNA is mir-35.

31. The method according to claim 28 wherein the tissue of interest is brain, liver, lung, or combinations thereof, the test sample comprises blood, the biological condition is brain cancer, cancer, liver cancer, lung cancer, or combinations thereof, and the target micro RNA is mir-16.

32. The method according to claim 28 wherein the tissue of interest is pancreas, the test sample comprises blood, the biological condition is pancreatic cancer, and the target micro RNA is mir-375.
